(19) United States
(12) Patent Application Publication
(10) Pub. No.: US 2025/0032733 A1
(43) Pub. Date: Jan. 30, 2025

(54) POSITIVE AIRWAY PRESSURE SYSTEMS AND METHODS FOR DELIVERING AIR TO A PATIENT

(71) Applicant: SLEEPRES, LLC, Murfreesboro, TN (US)

(72) Inventors: William NOAH, Pleasant View, TN (US); Bernard HETE, Kittanning, PA (US)

(21) Appl. No.: 18/696,808

(22) PCT Filed: Oct. 6, 2022
(86) PCT No.: PCT/US2022/045897

§ 371 Date: Mar. 28, 2024

Related U.S. Application Data
(60) Provisional application No. 63/253,027, filed on Oct. 6, 2021.
(60) Provisional application No. 63/348,257, filed on Jun. 2, 2022.
(60) Provisional application No. 63/398,775, filed on Aug. 17, 2022.

(51) Int. Cl. A61M 16/00

(52) U.S. Cl. CPC A61M 16/0003; A61M 16/022; A61M 2205/103; A61M 2205/3344; A61M 2205/36; A61M 2205/502; A61M 2205/7518

(57) ABSTRACT

A method for delivering air to a patient throughout the patient's respiratory cycle includes providing a positive airway pressure system with a flow generator and conduit having an outlet configured to connect to a patient's respiratory system. An inspiratory positive airway pressure is applied through the outlet during an inspiration phase of the patient's respiratory cycle, and an expiratory positive airway pressure is applied through the outlet during an expiration phase of the patient's respiratory cycle. The inspiratory positive airway pressure is less than the expiratory positive airway pressure.

### RELATED APPLICATIONS

This application claims priority from U.S. Provisional Application Ser. No. 63/253,027, filed Oct. 6, 2021, U.S. Provisional Application Ser. No. 63/348,257, filed on Jun. 2, 2022; and U.S. Provisional Application Ser. No. 63/398,775, filed on Aug. 17, 2022, the entire disclosures of which is incorporated herein by this reference.

### TECHNICAL FIELD

The presently-disclosed subject matter relates to positive airway pressure systems and methods for delivering air to a patient. In particular, the systems and methods of the present invention allow for the inspiratory positive airway pressure to be less than the expiratory positive airway pressure. In some instances, an air flow resistor is provided within a positive airway pressure system to reduce the air flow during the inspiration phase of the patient's respiratory cycle.

### BACKGROUND

Sleep apnea is a potentially serious disorder where breathing repeatedly stops and starts during sleep. Some estimates state that over 25 percent of adults between the ages of 30 and 70 years have sleep apnea, with that number rising to as high as 90 percent in some elderly males.

One of the most common treatments for sleep apnea is continuous positive airway pressure (CPAP) therapy. During therapy, a CPAP machine provides air flow through a mask or in-nose nasal pillow to increase air pressure in the patient's throat, which prevents their airway from collapsing. CPAP also increases functional residual capacity of the lung, which pulls on and stiffens the pharyngeal airway. Although CPAP therapy is effective for treating sleep apnea, the air flow provided through the mask often causes discomfort, which leads to patient's discontinuing use. Accordingly, there remains a need for articles and methods that more comfortably maintain pressure sufficient for treating sleep apnea.

### SUMMARY

The preset invention is, in part, directed to a method for delivering air to a patient throughout the patient's respiratory cycle. In some exemplary implementations of the present invention, the method includes providing a positive airway pressure system, the system including a flow generator and conduit having an outlet configured to connect to a patient's respiratory system. An inspiratory positive airway pressure is applied through the outlet during an inspiration phase of the patient's respiratory cycle, and an expiratory positive airway pressure is applied through the outlet during an expiration phase of the patient's respiratory cycle. According to the present invention, the inspiratory positive airway pressure is less than the expiratory positive airway pressure.

According to some exemplary embodiments, the positive airway pressure system further includes an air flow resistor to reduce air flow through the outlet during the inspiration phase of the patient's respiratory cycle thereby providing that the expiratory positive airway pressure is greater than the inspiratory positive airway pressure.

According to some exemplary embodiments, the air flow resistor is added to an existing positive airway pressure system configured to provide an inspiratory positive airway pressure greater than an expiratory positive airway pressure, the inclusion of the air flow resistor thereby modifying the operation of the existing positive airway pressure system.

According to some exemplary embodiments, the positive airway pressure system includes a computer with a processor for executing instructions stored in a memory component to modulate air flow through the outlet such that the expiratory positive airway pressure is greater than the inspiratory positive airway pressure.

According to some exemplary implementations, the patient sets the inspiratory positive airway pressure.

According to some exemplary implementations, the patient sets the expiratory positive airway pressure.

According to some exemplary implementations, the method further includes setting a maximum inspiratory positive airway pressure and a maximum expiratory positive airway pressure. The patient then sets the inspiratory positive airway pressure at or below the maximum inspiratory positive airway pressure.

According to some exemplary implementations, the patient sets the expiratory positive airway pressure at or below the maximum expiratory positive airway pressure.

According to some exemplary implementations, the inspiratory positive airway pressure is lower when the patient falls asleep and gradually increases.

According to some exemplary implementations, the inspiratory positive airway pressure is determined based on a patient parameter.

According to some exemplary implementations, the inspiratory positive airway pressure is determined based on an ambient condition.

According to some exemplary implementations, the inspiratory positive airway pressure is substantially zero.

According to some exemplary implementations, the expiratory positive airway pressure is between 4 and 20 cm H₂O.

According to some exemplary implementations, an average negative pressure provided by the patient during inspiration is determined and the inspiratory positive airway pressure is below the average negative pressure.

According to some exemplary embodiments of the present invention, a positive airway pressure system includes a flow generator, a conduit operably connected to the flow generator, the conduit having an outlet configured to connect to a patient's respiratory system. The flow generator provides an inspiratory positive airway pressure through the outlet during an inspiration phase of the patient's respiratory cycle and an expiratory positive airway pressure through the outlet during an expiration phase of the patient's respiratory cycle. According to the present invention, the inspiratory positive airway pressure is less than the expiratory positive airway pressure.

According to some exemplary embodiments, the positive airway pressure system further includes an air flow resistor to reduce air flow through the outlet during the inspiration phase of the patient's respiratory cycle thereby providing that the expiratory positive airway pressure is greater than the inspiratory positive airway pressure.

According to some exemplary embodiments, the air flow resistor additionally functions as a bacterial filter, humidifier, heat and moisture exchanger, or nebulizer.

According to some exemplary embodiments, a portion of the conduit is configured to function as the air flow resistor.

According to some exemplary embodiments, the positive airway pressure system includes a computer with a processor for executing instructions stored in a memory component to modulate air flow through the outlet such that the expiratory positive airway pressure is greater than the inspiratory positive airway pressure.

According to some exemplary embodiments, the system includes a patient interface configured to allow the patient to set the inspiratory positive airway pressure.

### BRIEF DESCRIPTION OF THE DRAWINGS

FIG. 1 a perspective view of an exemplary air resistor for inclusion in a positive airway pressure system;

FIG. 2 is a front view of the air resistor of FIG. 1;

FIG. 3 is a side view of the air resistor of FIG. 1;

FIG. 4 is a side sectional view of the air resistor taken along line 4-4 in FIG. 3;

FIG. 5 is a detailed view of one of the holes defined through the resistive element shown in FIG. 4;

FIG. 6 is a schematic representation of the operation of an exemplary positive airway pressure system of the present invention during the inspiration phase of the patient's respiratory cycle; and

FIG. 7 is a schematic representation of the operation of the exemplary positive airway pressure system during a portion of the expiration phase of the patient's respiratory cycle.

### DESCRIPTION OF EXEMPLARY EMBODIMENTS

Provided herein are systems and methods for providing variable pressure in airway pressure machines, such as CPAP and/or BiPAP machines. Unlike traditional systems and methods in which inspiratory positive airway pressure (“IPAP”) is greater than expiratory positive airway pressure (EPAP), according to the systems and methods of the present invention, IPAP is reduced to be lower than EPAP. Increased IPAP makes it initially difficult for patients to adopt CPAP treatment. It has been found, however, that this increased IPAP is not, in fact, as necessary for successful treatment as previously believed. Rather, while the “bilevel” concept is good for treating certain respiratory issues as well as obstructive sleep apnea (OSA), and contrary to the current understanding, according to methods of the present invention IPAP should be below EPAP for new patients. It has been found that this increases patient comfort without experiencing the same reduction in treatment as traditional CPAP or BiPAP machines in which IPAP is always greater than or equal to EPAP.

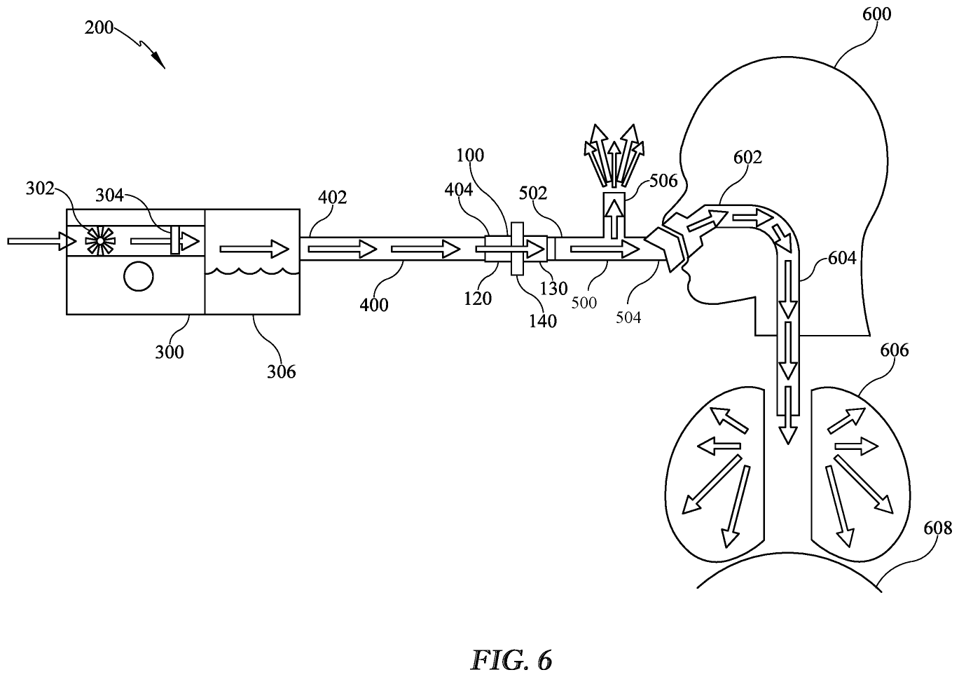

According to some exemplary embodiments, and referring to FIG. 6, an air pressure system 200 of the present invention includes a flow generator 300 which creates air flow through the system 200 to provide a desired air pressure to a patient 600. Specifically, the flow generator 300 includes a fan 302 which draws air in from the environment and through a flow meter 304. The air flow is then directed through a humidifier 306 before passing from the flow generator 300 into a first conduit 400. A second conduit 500 is also provided which has an outlet 504 operably connected to the patient's respirator system. The outlet 504 includes any suitable outlet for directing air provided by the flow generator 300 to the patient, such as, but not limited to, a full face mask, a partial face mask, a nasal pillow, or any other suitable outlet. The flow generator 300 illustrated in FIG. 6 is merely exemplary and may be any suitable device for providing a desired air pressure to the patient, such as, but not limited to, an air pump, a positive pressure device (e.g., CPAP), or any other suitable device.

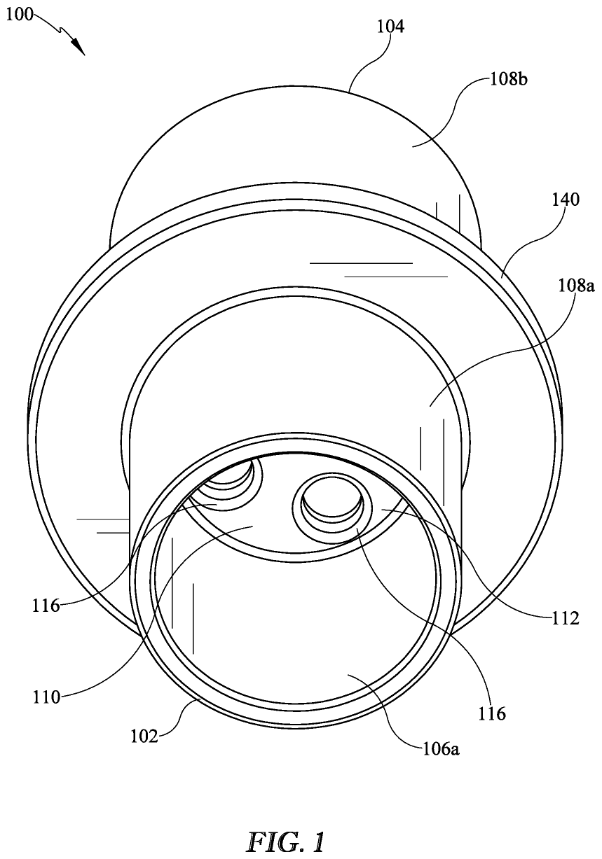

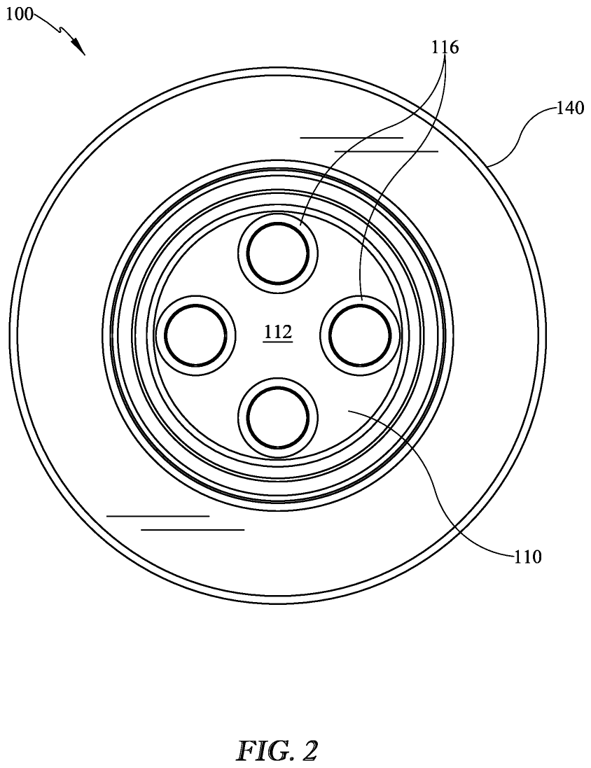

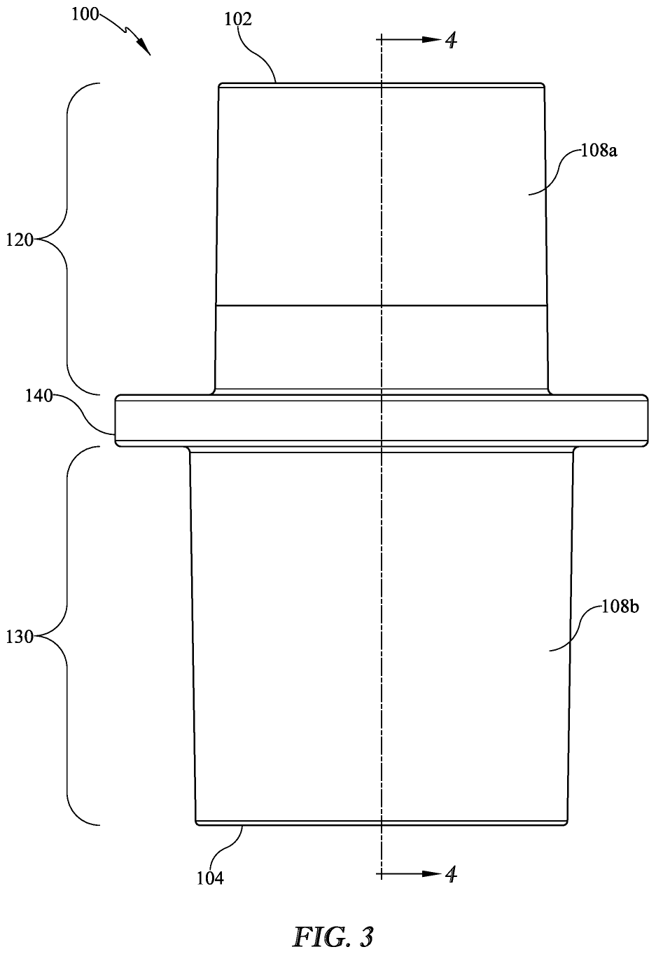

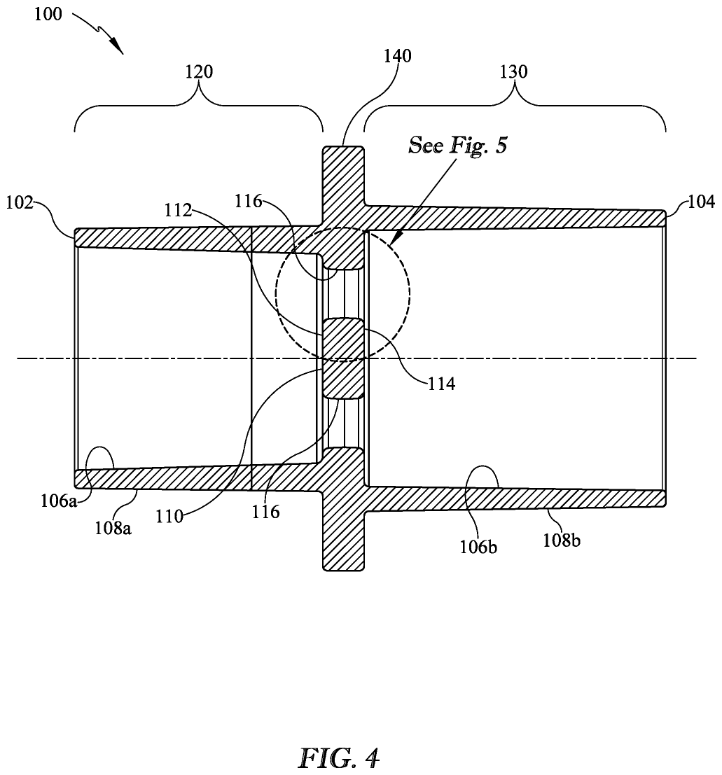

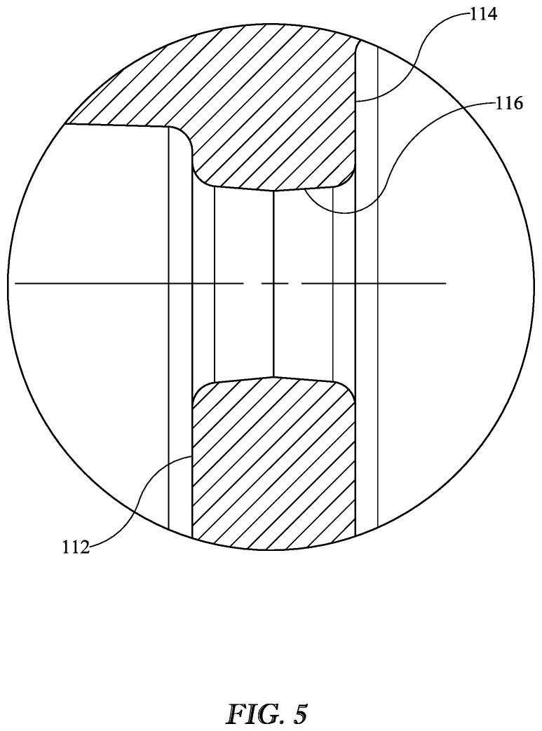

Turning now to FIGS. 1-5, the exemplary air flow resistor 100 is generally cylindrical with a first end 102 and a second end 104 opposite the first end 102. More specifically, the exemplary air flow resistor 100 includes an inlet side 120 that includes the first end 102 and an outlet side 130 that includes the second end 104. As perhaps best shown in FIG. 4, each of the inlet side 120 and the outlet side 130 has an inner surface 106a, 106b that collectively define an air channel through the air flow resistor 100. However, positioned between the inner surface 106a of the inlet side 120 and the inner surface 106b of the outlet side 130 is a resistive element 110 that interrupts the air channel. The resistive element 110 includes a first surface 112 facing the inlet side 120 of the air flow resistor 100 and a second surface 114 opposite the first surface 112 and facing the outlet side 130 of the air flow resistor 100. A plurality of holes 116 are defined by the resistive element 110 to extend therethrough from the first surface 112 to the second surface 114. In this exemplary embodiment, there are four holes 116 equally space around the resistive element 110 but the number, size, and placement of the holes is not limited. In fact, the overall configuration of the resistive element is not limited so long as it functions to limit the flow of air through the air flow resistor 100, as discussed further below. The air flow resistor 100 further includes a flange 140 extending outward from the outer surfaces 108a, 108b of the inlet side 120 and the outlet side 130 which facilitates its placement in the air pressure system 200.

The resistive element 110 may be formed from any material suitable for impeding the flow of air. Suitable materials include, but are not limited to, plastic, polymer, metal, ceramic, non-fabric, or a combination thereof. The exemplary air flow resistor 100 and resistive element 100 are integrally formed from K-Resin® KR03. As will be appreciated by those skilled in the art, the less material present in the opening (e.g., more holes, larger holes, the resistive element not fully extending across the opening, etc.) the less resistance provided by the resistive element.

Returning now to FIG. 6, in the exemplary air pressure system 200, the air flow resistor 100 is arranged and disposed to be positioned within the air flow path between the flow generator 300 and the outlet 504 of the second conduit 500. More specifically, the resistor 100 is positioned between the first conduit 400 and the second conduit 500. To this end, the first conduit 400 has a first end 402 operably connected to the flow generator 300 and a second end 404 connected to the inlet side 120 of the air flow resistor 100. Similarly, the second conduit 500 has a first end 502 connected to the outlet side 130 of the air flow resistor 100. The second conduit 500 further includes an exhaust port 506 located between the first end 502 and the outlet 504, as discussed further below. The specific configuration or location of the exhaust port 506 is not limited and may be integrated into a face mask, for example.

Although the system 200 shown in FIG. 6 has the air flow resistor 100 positioned between the first conduit 400 and the second conduit 500, additionally or alternatively, an air flow resistor may be positioned at or within the flow generator and/or the outlet, e.g., a face mask. For example, an air flow resistor could be positioned between the humidifier and the first conduit or between the flow generator and first conduit if no humidifier is present. In some embodiments, multiple resistors are positioned in the air flow path of the system. In such embodiments, each of the multiple resistors may have the same resistance or a different resistance as compared to each of the other resistors. The resistance of multiple resistors in series is additive, such that the total resistance of the system can be tailored by adding or removing resistors. Accordingly, in some embodiments, the number of resistors and/or the resistance of each resistor is selected to provide a desired resistance in the system. Furthermore, the location of the resistor or resistors can be anywhere in the gas flow path. Regardless, the inclusion of such resistors reduce the air flow through the outlet 504 during the inspiration phase of the patient's respiratory cycle thereby providing that the expiratory positive airway pressure is greater than the inspiratory positive airway pressure.

Without wishing to be bound by theory, it is believed that the resistor attenuates the increase in air flow, or adjusts the inspiratory time, as a patient breathes in (instantaneous flow), by decreasing the pressure at the patient airway in proportion to the flow rate through the resistor. Additionally or alternatively, in some embodiments, the resistor drops, decreases, or knocks down the peak inspiratory air flow of the system without, or with only minimally, dropping the baseline flow and expiratory flow of the system.

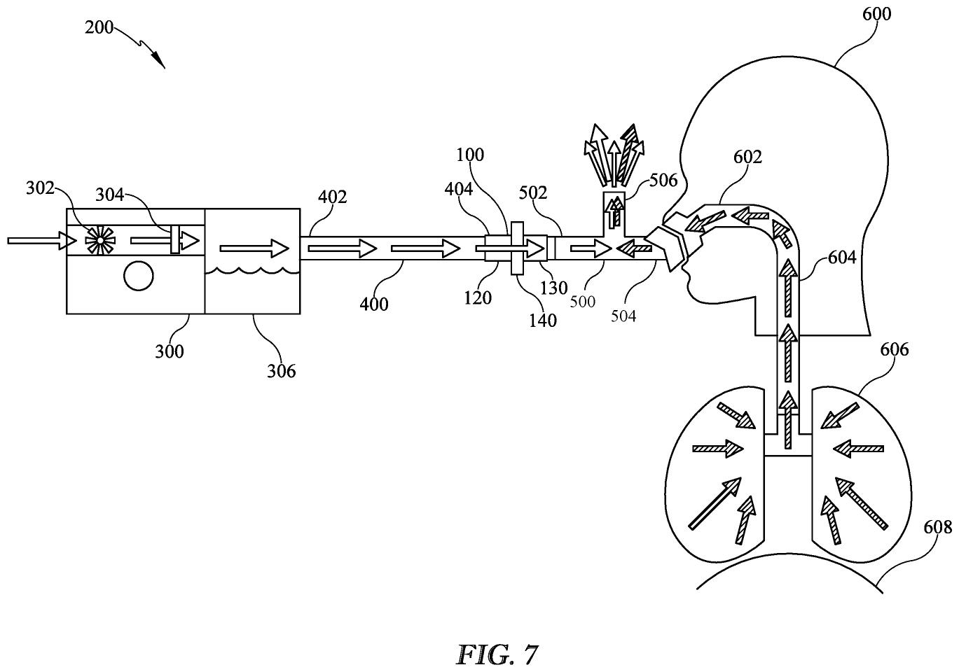

Referring still to FIG. 6, during an inspiration phase of the patient's breathing, the diaphragm 608 drops causing the lungs 606 to expand drawing air through the pharynx 604 and nasal passage 602. By comparison, and referring to FIG. 7, during an expiration phase of the patient's breathing, the diaphragm 608 and lungs 606 relax pushing air out through the pharynx 604 and nasal passage 602. Throughout both phases of the patient's respiratory cycle, the flow generator 300 provides air flow into the first conduit, thereby providing both an inspiratory positive airway pressure (IPAP) through the outlet 504 during the inspiration phase of the patient's respiratory cycle as well as an expiratory positive airway pressure (EPAP) through the outlet 504 during the expiration phase of the patient's respiratory cycle.

Typically, and as shown in FIG. 6, the air flow provided by the flow generator 300 and through the second conduit 500 is equal to the sum of the air flow demand from the patient and the air flow exiting through the exhaust 506, which is directly dependent on the pressure in the second conduit 500. Then, as shown in FIG. 7, both the air flow from the flow generator 300 as well as from the patient's exhaust both exit through the exhaust 506. As previously mentioned, inclusion of the air flow resistor 100 reduces the air flow through the outlet 504 during the inspiration phase of the patient's respiratory cycle.

More specifically, most existing positive airway pressure systems are designed with IPAP to be greater than EPAP. However, these existing positive airway pressure systems are designed with a predetermined amount of resistance calculated for the air flow from the flow generator 300 through the outlet of the first conduit 400 or even to the outlet 504. The additional resistance added by the inclusion of the air flow resistor 100 is not accounted for and is therefore not compensated for by the flow generator 300. As such, the inclusion of the air flow resistor 100 modifies the operation of the existing positive airway pressure system to provide EPAP which is greater than IPAP. Advantageously, the air flow resistor 100 is a passive device without any active controls and can therefore be added to an existing CPAP/BiPAP system.

According to some exemplary embodiment, the air flow resistor is provided in one of a variety of different forms that provide additional functionality. For example, tubing, mask, bacteria filters, HMEs (heat and moisture exchangers), nebulizer attachments, etc. can be modified to increase air flow resistance and function as a passive device similar to the inclusion of the air flow resistor 100 shown in the air pressure system in FIGS. 6 and 7.

However, according to other exemplary embodiments, rather than the inclusion of a passive resistor or other similar device, an air pressure system made in accordance with the present invention can also actively control the positive pressure delivered to a patient to provide pressure therapy where IPAP is less than EPAP during all, or some portion, of the inspiratory and expiratory phases. Not only does this provide adjustable control of the values for IPAP and EPAP between treatment periods, but active control allows for IPAP and/or EPAP to vary over a treatment period.

To this end, CPAP systems typically have a pressure sensor and/or flow sensor for controlling pressure and bulk flow of air into and through the conduits 400, 500. Such airway pressure systems include a computer with a processor for executing instructions stored in a memory component to modulate the air flow through the outlet. According to some exemplary embodiments, such active control is obtained via an algorithm which controls the device to allow IPAP to be less than EPAP, for example by adjusting the speed of the fan 302. In some embodiments, the algorithm reduces or completely turns off the compensation previously determined to overcome the expected pressure drop through the conduits 400, 500.

According to some implementations, treatment in which IPAP is less than EPAP is preferably used initially for new patients in order to acclimate the patients to positive pressure treatments. According to some other implementations, treatment in which IPAP is less than EPAP may continue indefinitely during treatment of the patient.

In some embodiments, the positive airway pressure system provides pressure during both the inspiratory and expiratory phases. The normal range of pressures (4-20 cm H₂O) are available during operation for both IPAP and EPAP, but it is contemplated that having an IPAP of zero, or substantially zero is possible as well. By reducing the pressure gradient between inspiration and expiration states, a patient should be more comfortable during treatment.

As previously mentioned, a primary advantage of the present invention which was unknown in the prior art is that IPAP is not therapeutically critical and could be considered needed only for comfort. According to some embodiments of the present invention, the patient is therefore able to adjust the IPAP level to maximize comfort, even if this adjustment brings IPAP to be lower than EPAP. In some embodiments, a maximum IPAP level is first set, and the patient is able to set the IPAP between a pressure from zero up to the maximum IPAP level. In some embodiments, the maximum IPAP level can be set so that a patient does not over pressurize himself. Alternatively, the maximum IPAP level could be specified by a clinician within the prescription and controlled at machine setup for the patient. Of course, a patient can also adjust the EPAP level in a similar manner.

In any event, it is contemplated that in some embodiments of the present invention, the setting chosen by the patient for IPAP and/or EPAP may only be the starting pressure of a treatment period. That is to say, the patient could dial in a comfort setting and the machine could increase the pressure after the patient falls asleep. Likewise, the IPAP and/or EPAP can gradually increase over multiple days to allow a new patient to more readily adapt to pressure therapy.

In fact, the IPAP and/or EPAP can be varied upwards and downwards throughout a treatment period depending on a variety of patient parameters and/or ambient conditions. Some possible patent parameters that may be used to adjust the IPAP and/or EPAP include, but are not limited to, tidal volume, breath rate, and minute volume. For example, in some exemplary implementations, if the tidal volume is large, the pressure level is gradually lowered to reduce the effect of pressure support so that hyperventilation is avoided. Some possible ambient conditions that may be used to adjust the IPAP and/or EPAP include, but are not limited to, ambient temperature, humidity, and atmospheric pressure. For example, in some exemplary implementations, when atmospheric pressure is low, e.g., when being used at a higher elevation, the pressure can be reduced to improve ventilation.

According to some embodiments, IPAP is adjusted in a manner to reduce the peak negative pressure draw by a patient as compared to the patient's normal tidal breathing. That is to say, a patent's average negative pressure drawn during inspiration is determined and the IPAP is set lower than this threshold value. Accordingly, the patient is partially assisted by the IPAP but is still required to provide some effort during inspiration.

Regardless of what level IPAP is set at, because IPAP is less than EPAP, there must be a pressure increase when transitioning from IPAP to EPAP. According to some embodiments, the pressure is increased up to the required EPAP therapy pressure during exhalation. However, in other embodiments, the pressure is increased partially or entirely during the inspiratory phase so that EPAP is at or near its maximum during most or all of the patient's expiratory phase. The increase in pressure can occur in a variety of patterns, including but not limited to, step, ramp, exponential, stair step, increasing oscillation, or any combination or variation of the above. The particular timing and pattern of pressure increase is not limited and can be determined based on the desired therapy and/or patient characteristics (e.g., age, gender, BMI, disease state, etc.).

One benefit to dropping IPAP lower than EPAP is increased patient comfort. As discussed above, IPAP is difficult for new patients on CPAP to tolerate, but it is known that EPAP is more important to stabilize the pharyngeal airway and prevent apnea. The system and methods of the present invention therefore reducing the discomfort experienced from IPAP while maintaining the benefits of EPAP.

Another benefit to the systems and methods described above in which the IPAP is reduced relative to EPAP (either passively or actively), is preventing increased ventilation and the resulting reduction in CO₂. The higher IPAP pressure provided by traditional CPAP/BiPAP machines can increase the tidal volume of ventilation so high that CO₂ gets too low and the brain stops telling the lungs to breath until the CO₂ evens back out. As such, up to 20% of new patients put on CPAP/BiPAP machines will experience treatment-emergent central sleep apnea (“TECSA”). Utilization of the system and methods of the present invention have been shown to reduce the risk of a patient developing TECSA.

Furthermore, because of the lower pressure on inhalation than exhalation, an exemplary system of the present invention will decrease the overall noise of the machine at night making it less disruptive to those sleeping in the room from an audible perspective. Previous CPAP machines typically “rev up” between the exhalation right before sending pressure back out for the inhalation. By reducing the pressure needed at inhalation, this “rev up” will be reduced, thereby decreasing the noise.

Further still, it is contemplated that in comparison to traditional CPAP/BiPAP devices, patients using the systems of the present invention will be able to keep their mouths closed more easily at night, which will avoid the discomfort of having to use a chinstrap or full face mask.

One of ordinary skill in the art will recognize that additional embodiments and implementations are also possible without departing from the teachings of the present invention or the scope of the claims which follow. This detailed description, and particularly the specific details of the exemplary embodiments disclosed herein, is given primarily for clarity of understanding, and no unnecessary limitations are to be understood therefrom, for modifications will become apparent to those skilled in the art upon reading this disclosure and may be made without departing from the spirit or scope of the claimed invention.

What is claimed is:

1. A method for delivering air to a patient throughout the patient's respiratory cycle, the method comprising:
providing a positive airway pressure system, the system including a flow generator and conduit having an outlet configured to connect to a patient's respiratory system;
applying an inspiratory positive airway pressure through the outlet during an inspiration phase of the patient's respiratory cycle; and
applying an expiratory positive airway pressure through the outlet during an expiration phase of the patient's respiratory cycle;
wherein the inspiratory positive airway pressure is less than the expiratory positive airway pressure.

2. The method of claim 1, wherein the positive airway pressure system further includes an air flow resistor to reduce air flow through the outlet during the inspiration phase of the patient's respiratory cycle thereby providing that the expiratory positive airway pressure is greater than the inspiratory positive airway pressure.

3. The method of claim 2, wherein the air flow resistor is added to an existing positive airway pressure system configured to provide an inspiratory positive airway pressure greater than an expiratory positive airway pressure, the inclusion of the air flow resistor thereby modifying the operation of the existing positive airway pressure system.

4. The method of claim 1, wherein the positive airway pressure system includes a computer with a processor for executing instructions stored in a memory component to modulate air flow through the outlet such that the expiratory positive airway pressure is greater than the inspiratory positive airway pressure.

5. The method of claim 1, wherein the patient sets the inspiratory positive airway pressure.

6. The method of claim 1, wherein the patient sets the expiratory positive airway pressure.

7. The method of claim 1, the method further comprising setting a maximum inspiratory positive airway pressure and a maximum expiratory positive airway pressure;
wherein the patient sets the inspiratory positive airway pressure at or below the maximum inspiratory positive airway pressure.

8. The method of claim 7, wherein the patient sets the expiratory positive airway pressure at or below the maximum expiratory positive airway pressure.

9. The method of claim 1, wherein the inspiratory positive airway pressure is lower when the patient falls asleep and gradually increases.

10. The method of claim 1, wherein the inspiratory positive airway pressure is determined based on a patient parameter.

11. The method of claim 1, wherein the inspiratory positive airway pressure is determined based on an ambient condition.

12. The method of claim 1, wherein the inspiratory positive airway pressure is substantially zero.

13. The method of claim 1, wherein the expiratory positive airway pressure is between 4 and 20 cm H₂O.

14. The method of claim 1, wherein an average negative pressure provided by the patient during inspiration is determined and the inspiratory positive airway pressure is below the average negative pressure.

15. A positive airway pressure system comprising:
a flow generator;
a conduit operably connected to the flow generator, the conduit having an outlet configured to connect to a patient's respiratory system;
wherein the flow generator provides an inspiratory positive airway pressure through the outlet during an inspiration phase of the patient's respiratory cycle and an expiratory positive airway pressure through the outlet during an expiration phase of the patient's respiratory cycle; and
wherein the inspiratory positive airway pressure is less than the expiratory positive airway pressure.

16. The system of claim 15, wherein the positive airway pressure system further includes an air flow resistor to reduce air flow through the outlet during the inspiration phase of the patient's respiratory cycle thereby providing that the expiratory positive airway pressure is greater than the inspiratory positive airway pressure.

17. The system of claim 16, wherein the air flow resistor additionally functions as a bacterial filter, humidifier, heat and moisture exchanger, or nebulizer.

18. The system of claim 16, wherein a portion of the conduit is configured to function as the air flow resistor.

19. The system of claim 16, wherein the positive airway pressure system includes a computer with a processor for executing instructions stored in a memory component to modulate air flow through the outlet such that the expiratory positive airway pressure is greater than the inspiratory positive airway pressure.

20. The system of claim 19, wherein the system includes a patient interface configured to allow the patient to set the inspiratory positive airway pressure.
